(19) United States
(12) Patent Application Publication
(10) Pub. No.: US 2011/0300625 A1
(43) Pub. Date: Dec. 8, 2011

(54) TISSUE FOR PROSTHETIC IMPLANTS AND GRAFTS, AND METHODS ASSOCIATED THEREWITH

(75) Inventors: David PANIAGUA, Houston, TX (US); R. David Fish, Houston, TX (US)

(73) Assignee: VELA BIOSYSTEMS LLC, Broomfield, CO (US)

(21) Appl. No.: 13/038,260

(22) Filed: Mar. 1, 2011

Related U.S. Application Data
(60) Provisional application No. 61/309,109, filed on Mar. 1, 2010.

(51) Int. Cl. C12N 5/071

(52) U.S. Cl. 435/325

(57) ABSTRACT

A prepared tissue for medical use with a patient is provided. Methods for preparing such tissue are also provided. Implantable tissue is provided by harvesting a tissue, such as but not limited to a pericardium tissue, and exposing the tissue to various cleaning, rinsing, treatment, separating, and fixation steps. The tissue of at least one embodiment is cleaned with distilled water, rinsed with isopropyl alcohol, and treated with a glutaraldehyde solution. The prepared tissue may be allowed to dry or partially hydrated prior to packaging and shipment. As such, the tissue can be implanted into the receiving patient in either a dry or wet state. The relatively thin yet strong tissue material is adapted for implanting within or grafting to human tissue. By way of example, the tissue may be used in a shunt, a valve, as graft material, as a patch, as a prosthetic tissue in a tendon and/or ligament, and a tissue product for wound management.

### CROSS-REFERENCE TO RELATED APPLICATIONS

The present application claims the benefit of U.S. Provisional Patent Application No. 61/309,109 filed on Mar. 1, 2010, the content of which is incorporated herein by reference in its entirety. Cross-reference is also made to U.S. patent application Ser. No. 10/887,688 filed on Jul. 10, 2004, which is a continuation-in-part application of U.S. patent application Ser. No. 10/037,266 filed on Jan. 4, 2002.

### FIELD

The present invention relates to the field of tissue engineering, and more particularly, to tissue for prosthetic implants and grafts.

### BACKGROUND

Preparing tissue for medical use to treat a patient is common. These tissues are typically used for implanting with or grafting to a human tissue. Prepared tissue is often used in shunts, tissue grafts and patches, as a prosthetic tissue in valves, tendon and/or ligament, and as tissue product for wound management. Many of these medical applications typically employ tissues obtained from mammalian animals and are thus termed xenografts. As with allografts (from human sources), xenograft tissue in the raw state contains immunologically “foreign” proteins and antigenic chemistry provocative of patient host immune responses that would cause destruction of implanted tissue as well as potentially harmful immune-mediated reactions. Thus, tissue for implantation in patients requires a number of preparatory chemical treatments to become biocompatible enough for implantation. For the preparation of xenograft tissue for structural applications, these treatments are typically directed to specific goals to isolate and preserve the structural proteins such as collagen: 1) remove cells within the tissue matrix, 2) remove unwanted chemical constituents, especially lipid components, and 3) chemically fix (i.e., cause thorough cross-linking of) structural proteins. Numerous manipulations of these and other steps in tissue processing have been employed with varying success in the art to achieve durable and biocompatible xenograft tissues for human implant. Nevertheless, conventional tissue materials are plagued by a variety of problems. For example, often in such applications, long-term function and survival of the tissue implants have been compromised by destructive inflammation, loss of structural integrity, and reactive calcification.

When using xenograft tissue membrane for use as formed sheet material, the tissue is usually cleaned and sterilized ex vivo, as outlined above. The preparation process itself can deteriorate the strength and biocompatibility characteristics of the tissue, or be the cause of latent host reactions that ultimately cause failure within the body. Often, the prepared tissue must maintain a certain thickness in order to have the desired strength traits. As such, the tissue material may be produced to be relatively thick, which may limit the manner of its application, and may also limit its biocompatibility.

Furthermore, in certain functional forms, such as for prosthetic heart valves, the prepared tissue must be stored in a liquid (usually a preservative) solution, otherwise the tissue will dry out and become brittle and prone to damage. Maintaining the tissue in a “wet” state adds mass and bulk to the tissue product since the moisture content of the tissue is higher and the volume of the tissue is greater when hydrated. Because the tissue must be stored “wet,” packaging must be robust to prevent leaks, the transportation environment must be carefully monitored and controlled, and once at the hospital or medical facility, significant efforts to rinse and prepare the tissue prior to use are needed.

By way of example and not limitation, when a surgeon is ready to use a bioprosthetic tissue heart valve, the valve and attached tissue must be rinsed, and in the case of transcatheter tissue heart valve devices, mounted onto a delivery system. In this example, if the tissue is associated with a percutaneously deliverable heart valve, the prosthetic heart valve is typically mounted to a balloon catheter in a catheterization lab. These steps extend procedure time, require manual manipulation of the tissue, and expose the tissue to harmful contaminants. Moreover, for the example of a percutaneously deliverable heart valve, human errors can be made in mounting and orienting catheters and sheaths.

Because the tissue has a relatively large profile, mass and volume, a surgeon's delivery options are often limited. For example, only patients having large enough vascular systems can use catheter-delivery procedures. Moreover, there is a need for tissue that can be used in a variety of medical indications unrelated to a percutaneously deliverable heart valves.

Accordingly, there is a need to address the shortcomings addressed above.

### SUMMARY

It is to be understood that the present invention includes a variety of different versions or embodiments, and this Summary is not meant to be limiting or all-inclusive. This Summary provides some general descriptions of some of the embodiments, but may also include some more specific descriptions of other embodiments.

Embodiments of the one or more present inventions include methods of preparing or treating tissue for medical use, as well as the actual tissue itself. Accordingly, in at least one embodiment, implantable tissue is provided by first harvesting a tissue, and thereafter treating the tissue by: (a) cleaning and decellularizing the tissue by rinsing and separating the tissue with distilled water; (b) optionally treating the tissue to additionally remove lipids by a glycerol pretreatment and exposure to light energy; (c) a secondary cleaning that includes a distilled water rinse, and rinsing with isopropyl alcohol; (d) final rinsing with distilled water; (e) fixation treating for collagen cross-linking by at least one of (I) immersion in formalin, (II) immersion in glycerol, (III) immersion in glutaraldehyde, (IV) immersion in glutaraldehyde filtered to limit oligomeric content, or (V) any of I-IV above with addition to the fixative solution of free amino acids lysine and/or histidine; (f) post-fixation treating by distilled water rinsing then isopropyl alcohol; and (g) final rinsing in distilled water. In at least one embodiment, the implantable tissue is then allowed to dry and thereafter is associated with a package for shipment. Alternatively, in at least one embodiment, the implantable tissue is then at least partially hydrated and associated with a package for shipment.

As noted above, one or more embodiments described herein are directed to one or more methods of preparing a section of tissue for medical use. By way of example and not limitation, the tissue may be used in a shunt, in a valve, as graft material, as a patch for repair of congenital heart defects, as a prosthetic tissue in tendon and/or ligament replacement, and a tissue product for wound management. Accordingly, a method of preparing a section of tissue for medical use is provided, the method comprising:

(a) cleaning and decellularizing the section of tissue by performing multiple rinses of the section of tissue with distilled water;

(b) rinsing the section of tissue with isopropyl alcohol for a first period of time of not less than about 7 days; and

(c) contacting the section of tissue with one of
(i) a formalin solution, or
(ii) a glutaraldehyde solution

for a second period of time of not less than about 6 days;

wherein step (b) occurs sometime after step (a), and wherein step (c) occurs sometime after step (b).

For the method directly above, in at least one embodiment, for step (c): if the formalin solution is used, then the formalin solution comprises a concentration of about 1-37.5% formalin, and more preferably, about 10% formalin; and if the glutaraldehyde solution is used, then the glutaraldehyde solution comprises a concentration of about 0.1-25% glutaraldehyde, and more preferably, about 0.25% glutaraldehyde.

In at least one embodiment, the method further comprises exposing the section of tissue to light energy for an exposure duration, the exposure duration extending until there is no further visible separation of lipid droplets from an exposed surface of the section of tissue. In at least one embodiment, the light energy is at least equivalent to exposing the section of tissue to a 25-100 watt light source, and more preferably, a 50 watt incandescent light source with a flat radiant face situated at a distance of about 10 centimeters from the exposed surface for about 15 minutes. In at least one embodiment, the method further comprises: (d) rinsing the section of tissue with distilled water and isopropyl alcohol for a post-fixation period of time of not less than about 7 days; wherein step (d) occurs after step (c). In at least one embodiment, the section of tissue comprises an ultimate tensile strength of greater than about 25 MegaPascals. In at least one embodiment, the section of tissue comprises a treated pericardium tissue.

In another embodiment, a method of preparing a tissue for medical use is provided, the method comprising: providing a section of tissue harvested from a mammalian organism; and causing osmotic shocking of the section of tissue by performing multiple rinses of the section of tissue with distilled water. In at least one embodiment, the method further comprises hydrating the section of tissue during a plurality of time intervals using distilled water. In at least one embodiment, the method further comprises not using saline for causing at least one of the osmotic shocking and the hydrating of the tissue. In at least one embodiment, the method further comprises pretreating the section of tissue with glycerol before contacting the section of tissue with one or more of isopropyl alcohol, glutaraldehyde and formalin. In at least one embodiment, the method further comprises contacting the section of tissue with a solution containing formalin after pretreating the section of tissue with glycerol. In at least one embodiment, the method further comprises contacting the section of tissue with a solution containing glutaraldehyde after pretreating the section of tissue with glycerol. In at least one embodiment, the method further comprises pretreating the section of tissue with isopropyl alcohol before contacting the section of tissue with either glutaraldehyde or formalin. In at least one embodiment, the method further comprises contacting the section of tissue with a solution containing formalin after pretreating the section of tissue with isopropyl alcohol. In at least one embodiment, the method further comprises contacting the section of tissue with a solution containing glutaraldehyde after pretreating the section of tissue with isopropyl alcohol. In at least one embodiment, the method further comprises exposing the section of tissue to light energy for a period of time, the period of time extending until there is no further visible separation of lipid droplets from an exposed surface of the section of tissue. In at least one embodiment, the light energy is at least equivalent to exposing the section of tissue to a 50 watt incandescent light source with a flat radiant face situated at a distance of about 10 centimeters from the exposed surface for about 15 minutes. In at least one embodiment, the section of tissue comprises a treated pericardium tissue.

Another embodiment of the one or more present inventions pertains to a method of preparing a section of tissue for medical use, comprising:

(a) contacting the section of tissue with distilled water;

(b) contacting the section of tissue with isopropyl alcohol for a pre-fixation period of time of not less than about 3 days; and

(c) contacting the section of tissue with one of
(i) a formalin solution, or
(ii) a glutaraldehyde solution for a fixation period of time of not less than about 3 days; and

(d) contacting the section of tissue with isopropyl alcohol for a post-fixation period of time of not less than about 3 days;

wherein step (b) occurs sometime after step (a), wherein step (c) occurs sometime after step (b), and wherein step (d) occurs sometime after step (c).

In at least one embodiment, for step (c): if the formalin solution is used, then the formalin solution comprises a concentration of about 1-37.5% formalin; and if the glutaraldehyde solution is used, then the glutaraldehyde solution comprises a concentration of about 0.1-25% glutaraldehyde. In at least one embodiment, for step (c): if the formalin solution is used, then the formalin solution comprises a concentration of about 8-12% formalin; and if the glutaraldehyde solution is used, then the glutaraldehyde solution comprises a concentration of about 0.1-0.5% glutaraldehyde. In at least one embodiment, the section of tissue comprises a treated pericardium tissue.

As mentioned above, one or more embodiments are directed to a tissue for medical use. Accordingly, a prepared tissue for medical use is provided, comprising: a section of treated tissue harvested from a mammalian organism, the section of tissue including an ultimate tensile strength of greater than about 15 MegaPascals. In at least one embodiment, the section of treated tissue has a thickness of between about 50 to 500 micrometers. In at least one embodiment, the section of treated tissue comprises a water content of less than about 60% by weight of the section of tissue. In at least one embodiment, the section of treated tissue comprises a water content of less than about 50% by weight of the section of treated tissue. In at least one embodiment, the section of treated tissue comprises a water content of less than about 40% by weight of the section of treated tissue. In at least one embodiment, the section of treated tissue is attached to a frame ex vivo for at least one of: (a) surgical use; or (b) percutaneous implantation. In at least one embodiment, the section of treated tissue does not include a matrix that has been exposed to a polymer infiltrate. In at least one embodiment, the section of treated tissue is unbraided and uncompounded (as used herein, “unbraided an uncompounded” means the tissue comprises a single layer and is not overlapped or otherwise intertwined). In at least one embodiment, the section of treated tissue comprises an ultimate tensile strength of greater than about 25 MegaPascals. In at least one embodiment, the section of treated tissue has been exposed to isopropyl alcohol before contacting the section of treated tissue with either glutaraldehyde or formalin. In at least one embodiment, the section of treated tissue has been exposed to a solution containing formalin after pretreatment with isopropyl alcohol. In at least one embodiment, the section of treated tissue has been exposed to a solution containing glutaraldehyde after pretreatment with isopropyl alcohol. In at least one embodiment, the section of treated tissue comprises a pericardium tissue.

In at least one embodiment, a prepared tissue for medical use with a patient is provided, comprising: a section of tissue harvested from a mammalian organism, wherein the section of tissue is prepared ex vivo for future grafting or implantation in the patient, the section of tissue including a thickness of about 50 to 500 micrometers and an ultimate tensile strength of greater than about 25 MegaPascals. In at least one embodiment, the section of tissue is unbraided and uncompounded. In at least one embodiment, the section of tissue comprises a water content of less than about 40% by weight of the section of tissue. In at least one embodiment, the section of tissue is attached to a frame ex vivo for at least one of: (a) surgical use; or (b) percutaneous implantation in the patient. In at least one embodiment, the section of tissue does not include a matrix that has been exposed to a polymer infiltrate. In at least one embodiment, the section of tissue comprises a treated pericardium tissue.

One or more embodiments described herein are directed to one or more articles comprising a treated tissue. Accordingly, an article is provided, comprising: a section of tissue harvested from an organism, the section of tissue residing within packaging, wherein the section of tissue is adapted for at least one of implanting within or grafting to a human tissue, and wherein the section of tissue comprises a water content of less than about 40% by weight of the section of tissue.

As used herein, the term “dry” (or “substantially dry”) when referring to the state of the tissue means a moisture content less than the water moisture content of the tissue when the tissue is allowed to fully rehydrate in the body of a patient. Typically, 70% by weight of the fully hydrated tissue membrane is water. Drying to a constitution of less than 40% by weight of water usefully alters the handling properties for purposes of folding, sewing or otherwise manipulating the tissue. As those skilled in the art will appreciate, the moisture content of the tissue may vary when dry. For example, the moisture content of the tissue when being folded and dry may be different than the moisture content of the tissue when dry and being shipped, for example, in a premounted state within a catheter delivery system.

With regard to delivery characteristics, another significant advantage of a prosthetic implant using a relatively thin tissue component described herein is that the prosthetic implant offers a relatively low packing volume as compared to commercially available prosthetic implants. In accordance with one or more embodiments, a dry tissue membrane has substantially less mass than a wet membrane. By way of example, a substantially dry pericardium tissue prepared by one or more of the present embodiments has approximately 30% of the mass of a wet pericardium tissue, and a marked reduction in profile and packing volume, thereby achieving a relatively low profile and making it suitable for implantation in greater number of patients.

Various components are referred to herein as “operably associated.” As used herein, “operably associated” refers to components that are linked together in operable fashion, and encompasses embodiments in which components are linked directly, as well as embodiments in which additional components are placed between the two linked components.

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

As used herein, “sometime” means at some indefinite or indeterminate point of time. So for example, as used herein, “sometime after” means following, whether immediately following or at some indefinite or indeterminate point of time following the prior act.

Various embodiments of the present inventions are set forth in the attached figures and in the Detailed Description as provided herein and as embodied by the claims. It should be understood, however, that this Summary does not contain all of the aspects and embodiments of the one or more present inventions, is not meant to be limiting or restrictive in any manner, and that the invention(s) as disclosed herein is/are understood by those of ordinary skill in the art to encompass obvious improvements and modifications thereto.

Additional advantages of the present invention will become readily apparent from the following discussion, particularly when taken together with the accompanying drawings.

### BRIEF DESCRIPTION OF THE DRAWINGS

To further clarify the above and other advantages and features of the one or more present inventions, a more particular description of the one or more present inventions is rendered by reference to specific embodiments thereof which are illustrated in the appended drawings. It is appreciated that these drawings depict only typical embodiments of the one or more present inventions and are therefore not to be considered limiting of its scope. The one or more present inventions is described and explained with additional specificity and detail through the use of the accompanying drawings in which:

FIG. 1 is a generalized flow chart illustrating preparation of tissue for use in an implantable construct or for use as a graft material;

FIGS. 2A-2B are flow charts illustrating elements of the tissue preparation;

FIG. 3 is a flow chart illustrating elements of the drying and sizing;

FIG. 4 is an elevation view of a piece of tissue; and

FIG. 5 is a graph that shows actual stress-strain test results for five tissue samples prepared in accordance with at least one embodiment.

The drawings are not necessarily to scale.

### DETAILED DESCRIPTION

Embodiments of the one or more inventions described herein include tissue for prosthetic implants and/or methods relating to preparation of tissue for prosthetic implants. A prosthetic implant made at least partially from tissue in accordance with at least one embodiment described herein can be surgically implanted or otherwise grafted to a patient. One or more embodiments of the prosthetic implant described herein have application for at least aortic and pulmonary valves, as well as in forming prosthetic ligaments and tendons.

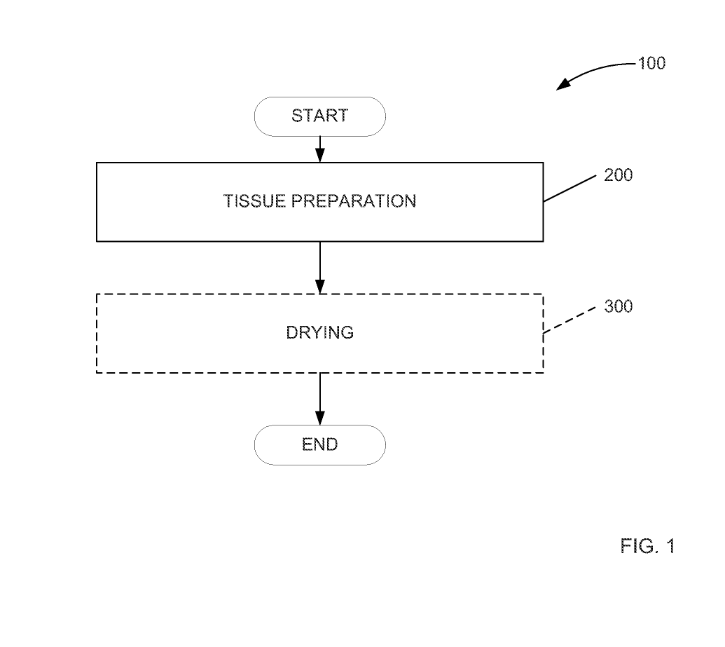

Referring now to FIG. 1, preparation of tissue for use in an implantable construct or as a graft is generally shown in method 100. Method 100 generally includes preparing the tissue at 200 and then, optionally, drying the tissue at 300 in preparation of manipulating the tissue for forming an implantable construct, such as a braided or folded structure. Further detail of the tissue preparation is provided below.

At least one or more embodiments described herein include a relatively thin tissue component. By way of example and not limitation, in at least one embodiment the tissue has a thickness of approximately 50-150 μm, and further possesses characteristics of pliability and resistance to calcification after implantation. The relatively thin nature of the tissue used in the implantable prosthetic implant assists with biocompatibility. In addition, the relatively thin tissue component thereby provides for a relatively low mass.

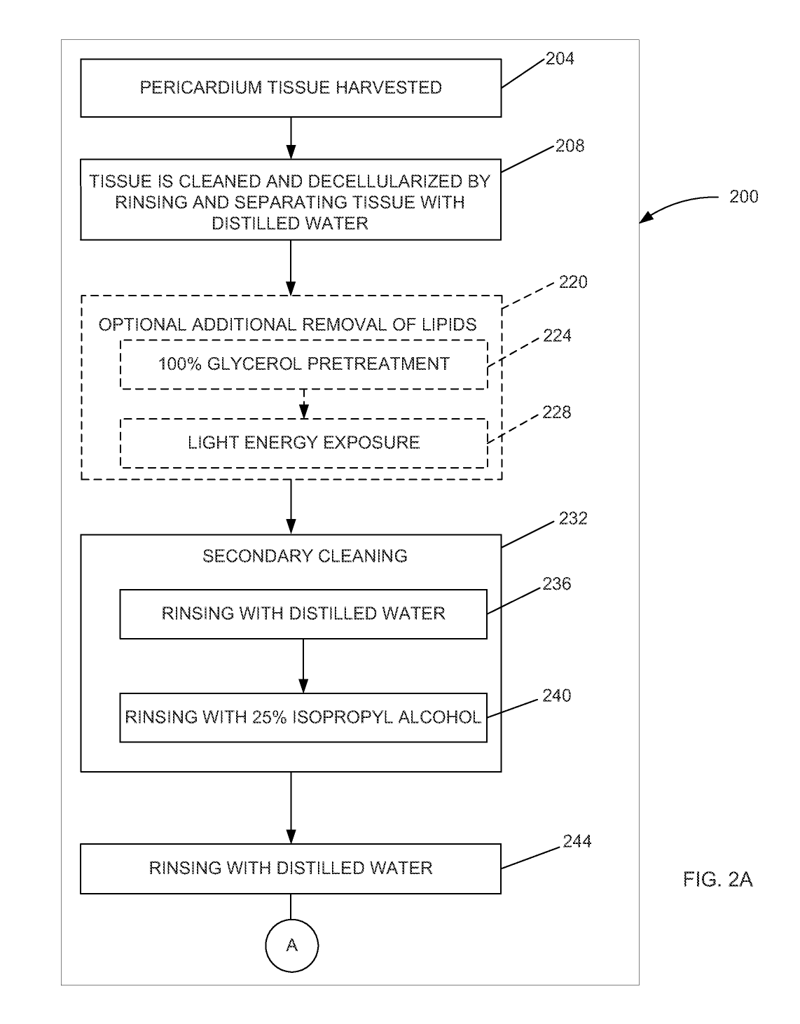

With reference now to FIG. 2A, the process associated with preparation of a biocompatible tissue consistent with the above-noted characteristics is described. In at least one embodiment, pericardium tissue, such as porcine or bovine pericardium tissue, is harvested at 204 and then processed to serve as biocompatible tissue. Accordingly, subsequent to the harvesting at 204, the pericardium tissue is cleaned and decellularized at 208. More particularly, in at least one embodiment the tissue is initially cleaned with distilled water using gentle rubbing and hydrodynamic pressure at 208 in order to remove adherent non-pericardial and non-collagenous tissue. In at least one embodiment, the hydrodynamic pressure at 208 is provided by spraying the tissue with a relatively weak stream of liquid to remove at least some of the non-collagenous material associated with the tissue. The rinsing at 208 is to achieve effective decellularization of the pericardium tissue through osmotic shock. Typically, the thickness of the tissue in the cleaned condition varies from about 50 to 500 micrometers, depending on the source of raw tissue. Cleaning preferably continues until there is no visible adherent non-pericardial or non-collagenous tissue.

With continued reference to FIG. 2A, after the tissue has been cleaned and decellularized at 208, the tissue then undergoes optional additional removal of lipids at 220 to further treat the tissue for preventing immunologic response and calcification. More particularly, the tissue first optionally undergoes a 100% glycerol pretreatment at 224 while being positioned on a flat surface (e.g., an acrylic plate), after which the tissue becomes nearly transparent.

At 228, the tissue optionally undergoes a “thermophotonic” process. In at least one embodiment, the tissue is optionally exposed to light energy for additional removal of lipids and for initial cross-linking of the collagen. By way of example and not limitation, in at least one embodiment a 25-100 watt incandescent light source, and more preferably, a 50 watt incandescent light source with a flat radiant face is employed at a distance of about 10 centimeters from the tissue surface, typically requiring 15 minutes of exposure before further visible separation of lipid droplets from the tissue stops.

Still referring to FIG. 2A, the tissue is then cleaned again in secondary cleaning at 232. More particularly, at 236 the tissue is again rinsed with distilled water. Thereafter, at 240 the tissue is rinsed with 25% isopropyl alcohol for periods of several hours to several days and weeks, depending on the desired tissue properties of pliability and tensile strength. By way of example, tissue prepared by the methods described herein has been successfully prepared by rinsing with 25% isopropyl alcohol for a period of 7 days, and after the further treatment steps described herein, provided an ultimate tensile strength of greater than 25 MegaPascals. In at least one embodiment where isopropyl alcohol is described as a rinsing agent, ethanol may be used in its place as an alternative, although resulting tissue properties may vary. Referring back to FIG. 2A, after the tissue is rinsed with isopropyl alcohol at 240, the tissue is then rinsed with distilled water at 244 as a final cleaning step and for rehydration.

Referring now to FIG. 2B, following the rinse with distilled water at 244, treatment of the tissue continues. More particularly, fixation for collagen cross-linking at 248 is achieved by performing at least one of the following:

At 248a, immersion of the tissue in 1-37.5% formalin, ideally a buffered solution, for between about 3 days to 5 weeks, and more preferably, for between about 3 days to 4 weeks, and more preferably yet, for between about 3 weeks to 4 weeks, at a temperature of between about 4 to 37° C., and more preferably, 10% formalin for 6 days at 20° C.; or

At 248b, immersion of the tissue in 100% glycerol for up to 6 weeks at between 4 to 37° C., and more preferably, immersion of the tissue in 100% glycerol for about 3 weeks at 20° C.; or

At 248c, immersion of the tissue in 0.1-25% glutaraldehyde for between about 3 days to 5 weeks, and more preferably, for between about 3 days to 4 weeks, and more preferably yet, for between about 3 weeks to 4 weeks, at 0 to 37° C., and more preferably, immersion of the tissue in 0.25% glutaraldehyde for 7 days at 4° C.; or

At 248d, immersion of the tissue in 0.1-25% glutaraldehyde (filtered to limit oligomeric content) for between about 3 days to 5 weeks, and more preferably, for between about 3 days to 4 weeks, and more preferably yet, for between about 3 weeks to 4 weeks, at 0 to 37° C., and more preferably, 0.25% glutaraldehyde for 7 days at 4° C.; or

At 248e, immersion in the tissue in one of the above formalin, glutaraldehyde, or oligomeric filtered glutaraldehyde solutions together with added amino acids, lysine and/or histidine, wherein the concentration of the amino acids, L-lysine or histidine, used as an additive to the fixative is in the range of about 100-1000 milliMolar, with a preferred value of about 684 mM.

In addition to the foregoing, combinations of the processes listed above may be performed, including: step a followed by step b; step a followed by step c; and step a followed by step d.

As those skilled in the art will appreciate, heat-shrink testing may be conducted on tissue samples to correlate the effectiveness of protein cross-linking. Here, results of heat-shrink testing performed on one or more samples of tissue prepared in accordance with at least one embodiment using formalin showed that the tissue had a shrink temperature of 90° C. This compares favorably with samples prepared using glutaraldehyde, wherein the shrink temperature was 80° C. Accordingly, formalin is a suitable variant of fixation. It is noted that formalin was generally abandoned by the field, largely because of material properties that were unfavorable and because of inadequate or unstable protein cross-linking. Such problems have been overcome through the pretreatments described herein, allowing production of tissue with strength, pliability, and durability in a relatively thin membrane. When used in a prosthetic implant, such as a heart valve, the tissue characteristics imparted by the tissue preparation process facilitate formation of a construct having a relatively low-profile, which also thereby facilitates dry packaging of the prosthetic implant. The same advantages are also achieved using the pretreatments when using a glutaraldehyde process.

Referring still to FIG. 2B, after fixation for collagen cross-linking at 248, an alcohol post-fixation treatment at 252 is preferably performed by rinsing the tissue in distilled water at 256, and then at 260 rinsing the tissue in 25% isopropyl alcohol for between about 30 minutes to 14 days or more at between about 0 to 37° C., and more preferably, for at least about 7 days at 20° C. At 264, the tissue undergoes a rinsing with distilled water.

In accordance with at least one embodiment, treatment of the tissue, including from the time of harvest to the time of implantation or grafting, does not include contact and/or exposure to a polymer to infiltrate and/or encapsulate tissue fibers of the tissue.

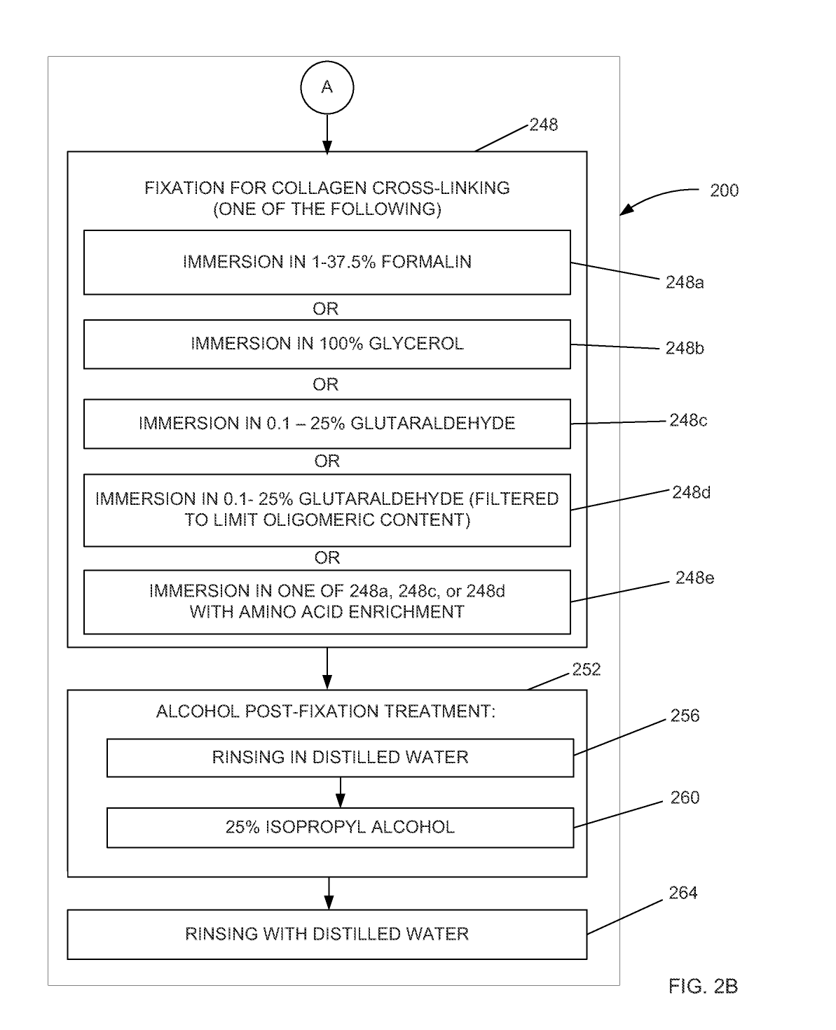

Referring now to FIG. 3, the drying process at 300 is performed after the tissue preparation at 200. Thus, in accordance with at least one embodiment, the tissue is dried under a load. More particularly, for the tissue drying at 304, the tissue is placed minimally stretched flat (that is, stretched just enough to eliminate visible wrinkles and bubbles) on a flat surface (e.g., a polymer or acrylic sheet) at 308, and held fixed at its edges at 312. Optionally, the joined tissue and underlying sheet are then set in a slight curve. The tension maintains the substantially flat structure of the tissue as it dries, thereby mitigating or preventing excessive shrinkage, wrinkling, and/or curling at the edges, and also making the rate of drying more uniform across the surface of the tissue because of the surface tension between the plate and the tissue. Alternatively, the tissue is dried while compressed between acrylic plates. When drying the tissue, the temperature is held at between about 4 to 37° C., and more preferably, between about 20 to 37° C. (i.e., approximately room temperature to normal human body temperature), and more preferably, at about 20° C. At 314, the drying process is performed in substantially dark conditions (i.e., substantially no visible light) for between about 6 hours to 5 days, and more preferably, for about 72 hours. By way of example, the tissue is dried in dark conditions at a temperature of about 20° C. for between about 6 hours to 5 days, and more preferably, for about 72 hours. As those skilled in the art will appreciate, drying the tissue while the tissue is compressed between plates requires a longer period of time.

In at least one embodiment, after drying, the tissue lots are inspected at 316, such as by stereomicroscopy, to identify and discard those with defects or discontinuities of the fiber matrix. If desired, the preferential fiber direction for each piece may be identified to determine a particular orientation, for example, to determine the free edge of the pieces that will form valve leaflets for a heart valve. Depending upon the size (i.e., the area) of the tissue being prepared and the size of tissue needed for a given implant, the tissue may be trimmed or otherwise sized in optional sizing at 320, such as by cutting the tissue into an appropriately sized and shaped sheet for implant formation and/or manipulation. Preferably, cutting of the tissue membrane is oriented so that the resulting free edge is parallel to the preferential fiber direction of the tissue membrane. Optionally, the free edge may also be cut with a parabolic or other curved profile to compensate for any attachment angles in order to increase the total contact surface between the tissue membrane and any associated frame or other structure. This approach minimizes weaknesses in the operating margins of the tissue assembly and advantageously distributes the principal loading forces of the operating implant along the long axis of the collagen fibers. As a result, the tissue is resistant to surface fracture and fraying.

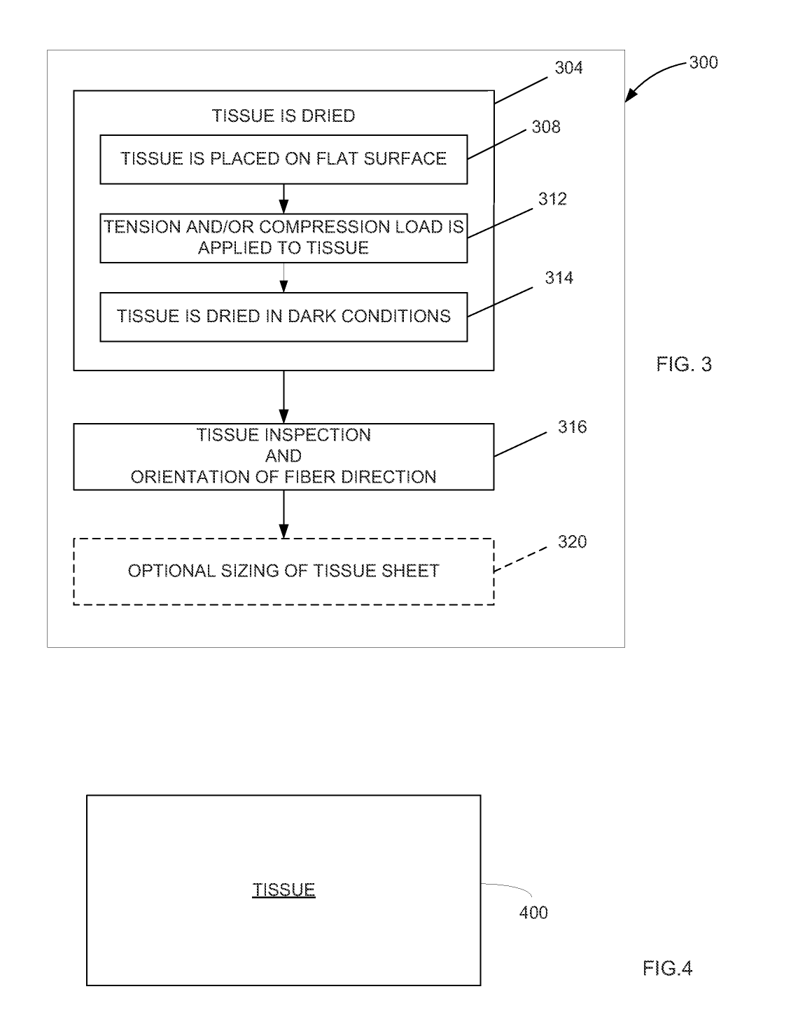

As shown in FIG. 3, optional sizing at 320 is performed after the drying at 304 and inspection at 316. A rectangular shaped piece of tissue 400 is shown in FIG. 4. The tissue 400 may be manipulated for use in a variety of prosthetic implants and grafts.

As mentioned above, tissue prepared by the methods described herein has been successfully prepared by rinsing with 25% isopropyl alcohol for a period of 7 days, and after the further treatment steps described herein, provided an ultimate tensile strength of greater than 25 MegaPascals. Here, the combination of tissue pliability and tensile strength is sought for purposes of producing a material having property characteristics suitable for being physically manipulated to form prosthetic implants, such as a tissue leaflet assembly for a heart valve or a ligament, while providing a tissue material that will operate properly once implanted. These techniques are intended to conserve and preserve collagen fibers, minimize damage to the tissue and improve tissue characteristics. The preparation and fixation techniques produce tissue membrane material that may be rendered and used at lesser thicknesses than typically rendered in the prior art. Thinner membranes are more pliable, but with conventional tissue preparation techniques the tensile strength of the tissue is sacrificed. Advantageously, the preparation techniques described herein have produced membranes that have as much as three times the tensile strength of a commercial product of the prior art. This achieved strength is thus desirable for providing a tissue assembly having a low profile with appropriate durability, even in a substantially dry state. More particularly, the tissue possesses a relatively high tensile strength. By way of example and not limitation, testing has shown that embodiments of tissue prepared as described herein provide a tissue having a tensile strength of approximately three times the tensile strength of current pericardial valve tissue, such as on the order of approximately 25 MegaPascals, thereby providing about 2,000 times the physiologic load strength for valve tissue. Moreover, testing of an embodiment of an implantable prosthetic heart valve made with tissue prepared as described herein and under a static load of greater than approximately 250 mmHg showed less than approximately 14% leakage, wherein such results are generally considered superior to surgical tissue valve prostheses.

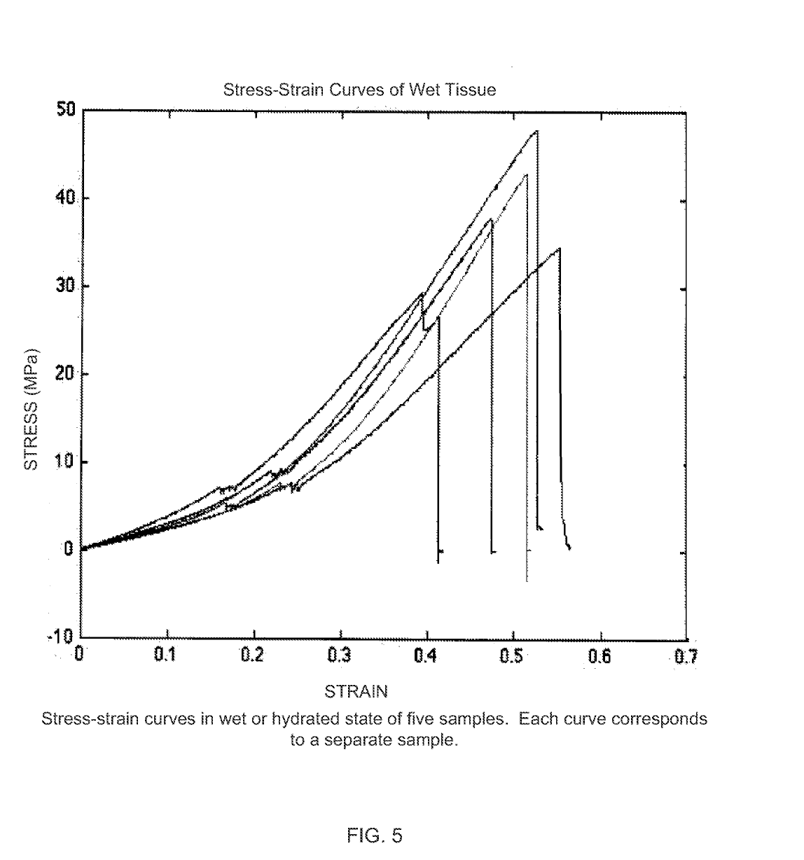

With reference to FIG. 5, stress-strain curve results for five different tissue samples prepared in accordance with an embodiment are shown. For the testing results shown, the yield stress or ultimate tensile strength was obtained by attaching strips of tissue fixed at the ends in a linear force tester and increasing the length by 0.3 mm/sec while recording resultant force (tension) until the material ruptured or separated entirely; these measurements were then used to calculate the stress-strain curves depicted in FIG. 5. As illustrated in the graph, the yield stress or ultimate tensile strength of the various tissue samples varied from about 30 to about 50 MegaPascals. More particularly, for each curve shown in FIG. 5, the testing procedures were the same. That is, each of the curves shown pertain to separate pieces of tissue that were subjected to the same test. The results show a minimum ultimate tensile strength of 30 MegaPascals, with a range up to 50 MegaPascals. Accordingly, the illustrated test results demonstrate consistency of the ultimate tensile strength results for the tissue treatment process.

It is to be understood that the tissue generated from one or more of the tissue preparation procedures described herein may be used for a variety of devices or uses, and that use in a prosthetic heart valve is but one possible application for utilizing the tissue. For example, the tissue may be used in a shunt, or as graft material for repair or modification of one or more human organs, including the heart and its blood vessels. By way of further example, the tissue may be used as a pericardial membrane patch for repair of congenital heart defects. The tissue also has application as a prosthetic tissue in tendon and ligament replacement, and as a tissue product for wound management. Moreover, for use in a prosthetic heart valve, the tissue may be configured in a variety of ways and attached to a frame in a variety of ways. In addition, a plurality of separate tissue pieces may each be connected together, such as by suturing, to form a larger composite of treated tissue material. Thereafter, whether the prosthetic implant or graft is made of a folded tissue assembly or a plurality of separate tissue pieces, the resulting prosthetic implant or graft may then be further manipulated for treatment of a patient.

In at least one embodiment, tissue generated from one or more of the tissue preparation procedures described herein may be used to form a prosthetic implant that includes a stent, frame, bone screw or other fastening or anchoring mechanism. In yet other embodiments, tissue generated from one or more of the tissue preparation procedures described herein may be used to form a prosthetic implant or graph that does not include a stent, frame, bone screw or other fastening or anchoring mechanism. Tissue generated from one or more of the tissue preparation procedures described herein may be may be packaged for delivery in a substantially dry, partially hydrated or hydrated (“wet”) state. For example, a prosthetic implant utilizing a prepared tissue described herein may be packaged for delivery as a hydrated prosthetic implant. Accordingly, while a portion of the tissue preparation process may include drying the tissue so that it may be manipulated more easily, the tissue may then be hydrated at a later point in time prior to implantation, and it may be maintained in a hydrated condition up to and including packaging, delivery and implantation into a patient. Hydration of the tissue membrane portion occurs rapidly and begins with simple preparatory flushing of the tissue. Those skilled in the art will appreciate that one or more embodiments described herein provide a tissue 400 suitable for implanting in a human, wherein the implantable tissue may be allowed to dry prior to implanting and effectively rehydrated at the time of implanting, such as by flushing of the tissue at the time of implanting using saline or water.

All embodiments described herein are described for use in human patients. However, all embodiments described herein have application for use in veterinary medicine, such as equine medicine.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

The one or more present inventions, in various embodiments, include components, methods, processes, systems and/or apparatuses substantially as depicted and described herein, including various embodiments, subcombinations, and subsets thereof. Those of skill in the art will understand how to make and use the present invention after understanding the present disclosure.

The present invention, in various embodiments, includes providing devices and processes in the absence of items not depicted and/or described herein or in various embodiments hereof, including in the absence of such items as may have been used in previous devices or processes (e.g., for improving performance, achieving ease and/or reducing cost of implementation).

The foregoing discussion of the invention has been presented for purposes of illustration and description. The foregoing is not intended to limit the invention to the form or forms disclosed herein. In the foregoing Detailed Description for example, various features of the invention are grouped together in one or more embodiments for the purpose of streamlining the disclosure. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the invention.

Moreover, though the description of the invention has included descriptions of one or more embodiments and certain variations and modifications, other variations and modifications are within the scope of the invention (e.g., as may be within the skill and knowledge of those in the art, after understanding the present disclosure). It is intended to obtain rights which include alternative embodiments to the extent permitted, including alternate, interchangeable and/or equivalent structures, functions, ranges or acts to those claimed, whether or not such alternate, interchangeable and/or equivalent structures, functions, ranges or acts are disclosed herein, and without intending to publicly dedicate any patentable subject matter.

What is claimed is:

1. A method of preparing a tissue for medical use, comprising:
providing a section of tissue harvested from a mammalian organism; and
causing osmotic shocking of the section of tissue by performing multiple rinses of the section of tissue with distilled water.

2. The method of claim 1, further comprising hydrating the section of tissue during a plurality of time intervals using distilled water.

3. The method of claim 2, further comprising not using saline for causing at least one of the osmotic shocking and the hydrating of the section of tissue.

4. The method of claim 1, further comprising pretreating the section of tissue with glycerol before contacting the section of tissue with one or more of isopropyl alcohol, glutaraldehyde and formalin.

5. The method of claim 4, further comprising contacting the section of tissue with a solution containing formalin after pretreating the section of tissue with glycerol.

6. The method of claim 4, further comprising contacting the section of tissue with a solution containing glutaraldehyde after pretreating the section of tissue with glycerol.

7. The method of claim 1, further comprising pretreating the section of tissue with isopropyl alcohol before contacting the section of tissue with either glutaraldehyde or formalin.

8. The method of claim 7, further comprising contacting the section of tissue with a solution containing formalin after pretreating the section of tissue with isopropyl alcohol.

9. The method of claim 7, further comprising contacting the section of tissue with a solution containing glutaraldehyde after pretreating the section of tissue with isopropyl alcohol.

10. The method of claim 1, further comprising exposing the section of tissue to light energy for a period of time, the period of time extending until there is no further visible separation of lipid droplets from an exposed surface of the section of tissue.

11. The method of claim 10, wherein the light energy is at least equivalent to exposing the section of tissue to a 50 watt incandescent light source with a flat radiant face situated at a distance of about 10 centimeters from the exposed surface for about 15 minutes.

12. The method of claim 1, wherein the section of tissue comprises a treated pericardium tissue.

13. A method of preparing a section of tissue for medical use, comprising:
(a) cleaning and decellularizing the section of tissue by performing multiple rinses of the section of tissue with distilled water;
(b) rinsing the section of tissue with isopropyl alcohol for a first period of time of not less than about 7 days; and
(c) contacting the section of tissue with one of
(i) a formalin solution, or
(ii) a glutaraldehyde solution
for a second period of time of not less than about 6 days;
wherein step (b) occurs sometime after step (a), and wherein step (c) occurs sometime after step (b).

14. The method of claim 13, wherein for step (c):
if the formalin solution is used, then the formalin solution comprises a concentration of about 1-37.5% formalin; and
if the glutaraldehyde solution is used, then the glutaraldehyde solution comprises a concentration of about 0.1-25% glutaraldehyde.

15. The method of claim 13, further comprising exposing the section of tissue to light energy for an exposure duration, the exposure duration extending until there is no further visible separation of lipid droplets from an exposed surface of the section of tissue.

16. The method of claim 15, wherein the light energy is at least equivalent to exposing the section of tissue to a 50 watt incandescent light source with a flat radiant face situated at a distance of about 10 centimeters from the exposed surface for about 15 minutes.

17. The method of claim 13, further comprising:
(d) rinsing the section of tissue with distilled water and isopropyl alcohol for a post-fixation period of time of not less than about 7 days;
wherein step (d) occurs sometime after step (c).

18. The method of claim 13, wherein the section of tissue comprises an ultimate tensile strength of greater than about 25 MegaPascals.

19. The method of claim 13, wherein the section of tissue comprises a treated pericardium tissue.

20. A method of preparing a section of tissue for medical use, comprising:
(a) contacting the section of tissue with distilled water;
(b) contacting the section of tissue with isopropyl alcohol for a pre-fixation period of time of not less than about 3 days; and
(c) contacting the section of tissue with one of
(i) a formalin solution, or
(ii) a glutaraldehyde solution for a fixation period of time of not less than about 3 days; and
(d) contacting the section of tissue with isopropyl alcohol for a post-fixation period of time of not less than about 3 days;
wherein step (b) occurs sometime after step (a), wherein step (c) occurs sometime after step (b), and wherein step (d) occurs sometime after step (c).

21. The method of claim 20, wherein for step (c):
if the formalin solution is used, then the formalin solution comprises a concentration of about 1-37.5% formalin; and
if the glutaraldehyde solution is used, then the glutaraldehyde solution comprises a concentration of about 0.1-25% glutaraldehyde.

22. The method of claim 20, wherein for step (c):
if the formalin solution is used, then the formalin solution comprises a concentration of about 8-12% formalin; and
if the glutaraldehyde solution is used, then the glutaraldehyde solution comprises a concentration of about 0.1-0.5% glutaraldehyde.

23. The method of claim 20, wherein the section of tissue comprises a treated pericardium tissue.
